(19) United States
(12) Patent Application Publication
(10) Pub. No.: US 2006/0013897 A1
(43) Pub. Date: Jan. 19, 2006

(54) Compositions for cancer therapy saponins or sapogenins

(76) Inventor: Dong Huang, Surrey (CA)

Correspondence Address: NEEDLE & ROSENBERG, P.C., SUITE 1000, 999 PEACHTREE STREET, ATLANTA, GA, 30309-3915, US

(21) Appl. No.: 10/517,749

(22) PCT Filed: Jun. 11, 2003
(86) PCT No.: PCT/CA03/00847

§ 371 Date: Aug. 23, 2005

Related U.S. Application Data
(60) Provisional application No. 60/435,305, filed on Dec. 23, 2002.

(30) Foreign Application Priority Data
Jun. 11, 2002 (CA) 2390290

(51) Int. Cl. A61K 31/56

(52) U.S. Cl. 424/725

(57) ABSTRACT

Compositions for cancer therapy comprising two or more saponins and/or sapogenins are provided. The compositions have enhanced anti-cancer activity when compared to each individual component alone. Also provided are pharmaceutical and non-pharmaceutical formulations comprising the compositions for use in the treatment of mammalian subjects having cancer, and pharmaceutical kits comprising such compositions or formulations.

### FIELD OF THE INVENTION

The present invention pertains to the field of cancer therapy and in particular to compositions comprising saponins and/or sapogenins for use in the treatment of cancer.

### BACKGROUND

There is a continuing need for development of new anti-cancer drugs and drug combinations. Accordingly, cancer research has been increasingly directed to the discovery of novel anti-cancer agents obtained from natural sources, as well as identifying and preparing synthetic compounds found in these natural sources.

Ginseng has long been recognized as a general tonic and a benign and safe herb. Many of the components of ginseng have been isolated and have been classified as: ginsenosides, carbohydrates, nitrogenous compounds, fat-soluble compounds, vitamins and minerals. The saponins derived from ginseng (also called “ginsenosides”) are believed to be the main active components of ginseng.

Saponins, in general, are composed of a sugar portion (glycon) and a non-sugar portion (aglycon or sapogenin). The sapogenins in ginseng, the backbone of saponins, are further classified into three types: protopanaxadiol and protopanaxatriol (which are tetracyclic terpenoids of the dammarane series), and oleanoic acid.

The sapogenin aglycon protopanaxatriol (aPPT) has the following chemical structures:

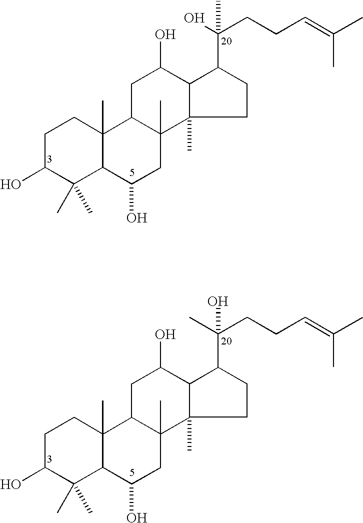

The sapogenin aglycon protopanaxadiol (aPPD) has the following chemical structure:

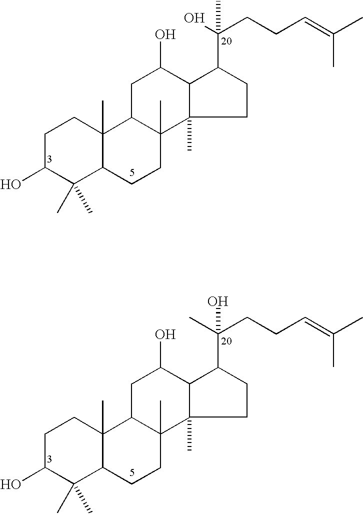

More than 25 dammarane-type saponins have been isolated from Panax ginseng C. A. Meyer, which vary in the number and type of monosaccharide residues present in the sugar side chains. The individual ginsenosides are named Rx according to their mobility on thin-layer chromatography plates. Examples of known ginsenosides include those in groups Ra through Rh. The isolation of three new dammarane-type saponins (named Rkl to Rk3) from heat-processed ginseng has also been reported recently (see Park, I. H. (2002) Arch Pharm Res. 25: 428-32).

In recent years, the beneficial effects of ginsenosides in the treatment of cancer has been reported. For example, U.S. patent application Ser. Nos. 09/910,887 (published as: 20030087835) and 09/982,018 (published as: 20030087836) describe novel sapogenin compounds having anti-cancer activities. Similarly, U.S. Pat. No. 5,776,460, reports ginsenoside Rh2 [3-O-β-D-glucopyranosyl-20(s)-protopanaxadiol] to have anti-cancer activities. Rh₂ is a saponin having the following chemical structure, in which “Glc” is the glycon (glucose):

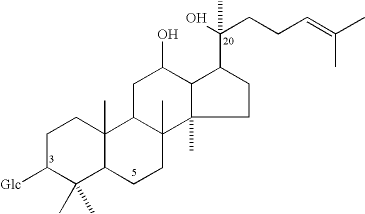

The literature also shows that Rh2 can induce differentiation and apoptosis of cancer cells (Kikuchi Y. et al. (1991) Anti-cancer Drugs. 2: 63-7; Lee K Y et al. (1996)

Cancer Lett. 110: 193-200; Oh M et al. (1999) Int J Oncol. 18: 869-75; Ota T et al. (1997) Life Sci. 60: PL39-88; Nakata H et al. (1998) Jpn J Cancer Res. 89: 733-80; Kim H. E et al. (1999) Life Sci. 65: PL33-80; Park JA et al. (1997) Cancer Lett. 121: 73-81); inhibit the growth of human ovarian cancer in nude mice after oral administration (Nakata H et al. (1998) Jpn J Cancer Res. 89: 733-80); and kill multi-drug resistant (MDR) cancer cells in vitro, when used in combination with other chemotherapy drugs (U.S. Patent Application No. 60/204,785).

The ginsenoside Rg3 [3-O-[β-D-glucopyranosyl (1→2)-β-D-glucopyranosyl]-20(s)-protopanaxadiol] has also been studied and reported to inhibit the activity of various cancer cells (Shinkai K et al (1996) Jpn J Cancer Res. 87: 357-62), including, human prostate cancer cells in vitro (Liu WK et al. (2000) Life Sci. 67(11): 1297-306), lung metastasis in mice (Iishi H et al. (1997) Clin Exp Metastasis 15: 603-11), and peritoneal metastasis in rats (Mochizuki M et al. (1995) Biol Pharm Bull. 18: 1197-202). Additionally, the two sapogenins, protopanaxadiol and protopanaxatriol, have been reported to chemosensitize MDR cancer cells in vitro, when used in combination with other chemotherapy drugs (U.S. patent application Ser. No. 09/957,082 (published as: 20030092638).

In addition to the anti-cancer activity of certain isolated ginsenosides, some oleanane-type pentacyclic terpenoid compounds isolated from ginseng have been shown to have some anti-carcinogenic activity in in vitro assays (Shibata, S. (2001) J. Korean Med. Sci., 16 Suppl.: S28-37).

This background information is provided for the purpose of making known information believed by the applicant to be of possible relevance to the present invention. No admission is necessarily intended, nor should be construed, that any of the preceding information constitutes prior art against the present invention. Publications referred to throughout the specification are hereby incorporated by reference in their entireties in this application.

### SUMMARY OF THE INVENTION

An object of the present invention is to provide a composition for cancer therapy and the use of this composition in the treatment of cancer. In accordance with an aspect of the present invention, there is provided a composition, comprising an activity-enhancing amount of two or more saponins and/or sapogenins, and having anti-cancer activity.

In accordance with another aspect of the invention, there is provided a pharmaceutical formulation for the treatment of cancer, comprising a therapeutically effective amount of the composition and a suitable carrier.

In accordance with another aspect of the invention, there is provided a non-pharmaceutical formulation for the treatment of cancer, comprising a therapeutically effective amount of the composition and a suitable carrier.

In accordance with another aspect of the invention, there is provided a use of the composition for the treatment of a mammalian subject having cancer.

### BRIEF DESCRIPTION OF THE FIGURES

FIG. 1 provides a graphical representation of the cell viability of human glioma tumor cells (U87) treated with various individual saponins and sapogenins and a composition of the present invention;

FIG. 2 provides a graphical representation of the cell viability of mouse melanoma cells (B16) treated with various individual saponins and sapogenins and a composition of the present invention;

FIG. 3 provides a graphical representation of the cell viability of human breast cancer cells (MCF7) treated with various individual saponins and sapogenins and a composition of the present invention;

FIG. 4a provides a graphical representation of the cell viability of pancreatic cancer cells (BXCP-3) treated with various concentrations of a composition of the present invention;

FIG. 4b provides a graphical representation of the cell viability of pancreatic cancer cells (CAPAN-1) treated with various concentrations of a composition of the present invention; and

FIG. 5 demonstrates the in vivo cancer inhibitory effect of various concentrations of a composition of the present invention.

### DETAILED DESCRIPTION OF THE INVENTION

The present invention is based on the finding that a combination comprising saponins and/or sapogenins, which are known individually to have anti-cancer activities, has a higher cytotoxic effect on cancer cells than any of the individual components alone, i.e., an unexpected enhanced cytotoxic effect is observed when the combination is used to treat cancer cells.

Thus the compositions of the present invention demonstrate a higher anti-cancer efficacy and potency than each of the individual components of the composition when used alone. This level of anticancer efficacy and potency would be achievable only by the use of extremely high doses of the individual components, which would be intolerable due to adverse toxic side effects.

Definitions

Unless defined otherwise, all technical and scientific terms used herein have the same meaning as commonly understood by one of ordinary skin in the art to which this invention pertains.

“Extract,” as used herein, means a composition physically or chemically extracted from plant material, which comprises one or more saponins and/or sapogenins. A person of skill in the art will appreciate that an extract may be produced following methods well known in the art as provided herein, for example by contacting a solvent with plant material. The extract may be a crude extract, or it may be a partially purified extract.

“Plant material,” as used herein, means one or more part of a plant taken individually or in a group, and includes, but is not restricted to, leaves, flowers, roots, seeds, stems, berries and the like.

“Crude extract,” as used herein, refers to an extract from a plant in its raw, unprocessed form.

“Partially purified,” when used herein with reference to an extract from a plant, means that the extract is in a form that contains fewer proteins, nucleic acids, lipids, carbohydrates or other similar materials with which it is naturally associated in the plant than are contained by a crude extract. The extract(s) used in the compositions of the invention are partially purified.

“Active components,” as used herein, means those components of the composition to which the anti-cancer activity of the composition is attributed. The active components of the compositions of the present invention include saponins and/or sapogenins.

“Saponin,” as used herein, means a plant glycoside that can be digested to yield a sugar and a sapogenin aglycon. Saponins are also referred to as “ginsenosides.”

“Sapogenin,” as used herein, means a nonsugar portion of a saponin that is typically obtained by hydrolysis, and which has either a complex terpenoid or a steroid structure.

“Enhanced effect,” as used herein with reference to a combination of two or more active components indicates that the combined activity of the two or more components exceeds the activity of each component alone.

“Activity-enhancing amount/activity-enhancing proportion,” as used herein, indicates the amount of one component of a composition of the present invention that is sufficient to produce an enhanced effect, such as an enhanced cytotoxic action, when used in combination with another component or components of the composition.

“Efficacy,” as used herein, means the cancer cell killing effect of a component or composition.

“Potency,” as used herein, means the efficacy of a component or composition relevant to dose.

“Drug resistant cancer,” as used herein, refers to an innate or acquired ability of cancer cells and/or a tumor to develop resistance to treatment. The term “drug-resistant cancer” includes multi-drug resistant (MDR) cancers. By convention: primary MDR cancer is untreated cancer that is considered unresponsive to chemotherapy and secondary MDR cancer develops resistance during the course of treatment. One skilled in the art will appreciate that drug resistance may be caused by several mechanisms including, but not limited to, decreased drug accumulation (e.g., active excretion of the chemotherapeutic by a protein pump (e.g., P-glycoprotein)), accelerated metabolism of the drug and other alterations of drug metabolism, and an increase in the ability of the cell to repair drug-induced damage. In the context of the present invention, the term “drug resistant cancer” is intended to encompass cancers that exhibit resistance to one chemotherapeutic drug, or to more than one chemotherapeutic drug, regardless of the mechanism of action of the resistance.

“Advanced cancer,” as used herein, refers to overt cancer in a subject, wherein such overt cancer is not localized, and is not amenable to cure by local modalities of treatment, such as surgery or radiotherapy.

“Primary cancer,” as used herein, refers to the original tumor or primary tumor and is usually named for the part of the body in which it originates.

“Metastatic cancer,” as used herein, refers to a cancer which has spread from an initial site (“primary cancer”) to another site(s) (“secondary cancer”). Virtually all cancers can become metastatic. The term, thus, is not limited to any one particular type of cancer. Metastatic cancers are frequently drug resistant.

“Recurrent cancer,” as used herein, refers to a reappearance of a cancer that was thought to be cured or inactive (i.e., in remission). A cancer may recur after several weeks, several months, a few years, or many years. One skilled in the art will understand that a recurrent cancer starts from cancer cells that were not removed or destroyed by the original therapy, e.g. chemotherapy, radiotherapy, surgery.

Compositions of Saponins and/or Sapogenins

The present invention provides for compositions comprising two or more saponins and/or sapogenins in combination for the treatment of cancer in a mammalian subject. The compositions of the present invention can comprise, for example, two or more saponins, two or more sapogenins, or one or more saponins together with one or more sapogenins. In accordance with the present invention, the compositions demonstrate an enhanced effect in the inhibition of growth of cancer cells compared to the effects of each of the components alone. The enhanced effect of the compositions may or may not be synergistic.

The combinations of the present invention show an increased efficacy of at least about 5%, when compared to the efficacy of each of the components alone. In one embodiment, the combinations show an increased efficacy of at least about 10%, when compared to the efficacy of each of the components alone. In another embodiment, the combinations show an increased efficacy of at least about 15%. In a further embodiment, the combinations show an increased efficacy of at least about 20%. In other embodiments, the combinations show an increased efficacy of at least about 23%, at least about 25%, at least about 27% and at least about 30%.

The saponins and sapogenins for use in the compositions are those that can be derived from plants of the genus Panax. Suitable sapogenins, therefore, are protopanaxadiol and propanaxatriol. In one embodiment of the present invention, the compositions comprise one or more of the sapogenins aglycon protopanaxadiol (aPPD) and aglycon protopanaxatriol (aPPT).

A large number of saponins derived from plants of the genus Panax are known in the art and are suitable for use in the compositions of the present invention. The saponins may be protopanaxadiol-type saponins or propanaxatriol-type saponins. Examples of suitable saponins include, but are not limited to, Ra₁, Ra₂, Rb₁, Rb₂, Rb₃, Rc, gluco-, R_{c}, Rd, Re, Rf, 20-gluco-Rf, Rg₁, Rg₂, Rg₃, Rh₁, Rh₂, Rh₄, notoginsenoside R₁, koryoginsenoside R₂, Rs₁, Rs₂, Q-R₁ and chikusetsusaponin V. One skilled in the art will appreciate that, while the natural form of most ginsenosides is 20S, during the process of extracting the ginsenosides from plant material, 20R compounds may be formed. The present invention thus contemplates the use of saponins in either the 20S and 20R conformation.

In one embodiment of the present invention, the compositions comprise one or more protopanaxadiol-type saponin and/or one or more protopanaxatriol-type saponin. In another embodiment, the compositions comprise the saponin Rh₂. In a further embodiment, the compositions comprise the saponin Rg₃. In a specific embodiment, the composition comprises a combination of saponins and/or sapogenins selected from the group: Rh2, aPPT, and aPPD. In another specific embodiment, the composition comprises a combination of saponins and/or sapogenins selected from the group: Rh2, Rg3, aPPT, and aPPD. The compositions may additionally comprise minor amounts of one or more unspecified saponins.

The amount of each component to be included in the compositions varies between about 1 and about 90% (w/w). In one embodiment of the invention, the amount of each saponin in the composition is between about 1 and about 50% (w/w) and the amount of each sapogenin is between about 5 and about 75% (w/w). In another embodiment, the amount of each saponin in the composition is between about 5 and about 40% (w/w) and the amount of each sapogenin is between about 10 and about 70% (w/w).

An exemplary composition of the present invention thus comprises 1-90% (w/w) Rh2, 1-90% (w/w) aPPT, and 1-90% (w/w) aPPD. In one embodiment, the composition comprises 1-50% (w/w) Rh2, 5-40% (w/w) aPPT, and 5-75% (w/w) aPPD. In another embodiment, the composition comprises 5-40% (w/w) Rh2, 10-35% (w/w) aPPT, and 10-70% (w/w) aPPD. In a further embodiment, the composition comprises 7-42% (w/w) Rh2, 15-30% (w/w) aPPT and 40-70% (w/w) aPPD.

The saponins and sapogenins for use in the compositions of the invention may be obtained, individually or in combination, from extracts prepared from plants of the genus Panax. Many saponins and/or sapogenins are also available commercially (Pegasus Pharmaceuticals, Inc., Richmond, British Columbia, Canada) or can be chemically or biologically synthesized using techniques well known to persons of skill in the art (see, for example, Shibata, S. (2001) J. Korean Med. Sci., 16 Suppl.: S28-37, and references therein).

The compositions of the present invention can be prepared directly from a partially purified plant extract by simple dilution to an appropriate concentration, or one or more individual saponin and/or sapogenin components can be added to a plant extract in order to adjust the proportion of active components to the desired amount. Alternatively, the compositions can be prepared by mixing two or more individual saponin and/or sapogenin components in activity-enhancing proportions. The individual saponins and sapogenins can be purified or partially purified.

Plant Material

In one embodiment of the present invention, the saponins and/or sapogenins are extracted from plant material. In another embodiment, a partially purified plant extract comprising a combination of saponins and/or sapogenins in activity-enhancing proportions is used to prepare the composition.

In accordance with the present invention, plant material suitable for preparing extracts is derived from plants of the genus Panac Examples of plants from which plant material may be obtained include, but are not limited to, Panax aureus; Panax bipinnatifidus (also known as: Panax major and Panax pseudoginseng var. bipinnatifidus); Panax ginseng C. A. Meyer (also known as: Panax schinseng); Panax japonicus (also known as: Panax pseudoginseng subsp. japonicus; Panax pseudoginseng var. japonicus and Panax repens); Panax notoginseng (also known as: Aralia quinquefolia var. notoginseng and Panax pseudoginseng var. notoginseng); Panax pseudoginseng (also known as: Aralia pseudoginseng and Panax pseudoginseng var. pseudoginseng); Panax wangianus (also known as: Panax pseudoginseng var. wangianus); Panax quinquefolius, Panax stipuleanatus; Panax trifolius; Panax vietnamensis and Panax zingiberensis.

Plant material that may be used in the present invention includes one or more plant parts taken individually or in a group and may include, but is not restricted to, leaves, flowers, roots, seeds, berries, stems and parts thereof. As is known by persons skilled in the art, the chemical composition and efficacy of a plant extract varies with the phenological age of the plant, percent humidity of the harvested material, the plant parts chosen for extraction, and the method of extraction. Methods well-known in the art can be adapted by a person of ordinary skill in the art to achieve the desired yield and quality of the extract

In one embodiment of the present invention, plant material derived from Panax ginseng is used to prepare extracts containing saponins and/or sapogenins In another embodiment, the plant material is derived from Panax quinquefolium L. In a further embodiment, the plant material is derived from Panax notoginseng.

Pre-Treatment of Plant Material

In addition to such parameters as the phenological age of the plant, the region or area that the plant is harvested from, the percent humidity of the harvested material, the plant parts chosen for extraction, and the method of extraction, the chemical composition of an extract may be affected by pre-treatment of the plant material. For example, when a plant is stressed, several biochemical processes are activated and many new compounds, in addition to those constitutively expressed, are synthesized as a response. In addition to pests, fungi, and other pathogenic attacks, stressors include drought, heat, water and mechanical wounding. Stressing a plant may, therefore, be used as a tool to increase the content of one or more desired compounds. A person of skill in the art will recognize that one of the above stressors may be employed for this purpose, or various combinations of stressors may be used. For example, the effects of mechanical wounding can be increased by the addition of compounds that are naturally synthesized by plants when stressed. Such compounds include jasmonic acid (JA). Analogs of oral secretions of insects can also be used in this way to enhance the reaction of plants to stressors.

In one embodiment, the compositions of the present invention are derived from extracts of plant material which have been pre-treated, for example by stressing the plant by chemical or mechanical wounding, drought, heat, or cold, or a combination thereof, before plant material collection and extraction.

Preparation of the Plant Extract and Validation of Active Components

Extracts can be prepared from plant material by standard techniques known in the art. A variety of strategies are available for preparing extracts from plant material, the choice of which depends on the ability of the method to extract the components required for the compositions of the present invention. Examples of suitable methods for preparing extracts include, but are not limited to, hydro-distillation, direct steam distillation, solvent extraction, and Microwave Assisted Process (MAP™).

Thus, for example, the plant material can be treated by boiling in water. The components are released into the water and are subsequently recovered after distillation and cooling. Alternatively, the plant material can be treated with steam, which causes the components within the cell membranes to diffuse out and form a mixture with the water vapor. The steam and components can then be condensed and the extract collected. Organic solvents can also be used to extract the active components. Non-limiting examples of such organic solvents include methanol, ethanol, hexane, and methylene chloride. As an alternative to aqueous or organic solvents, microwaves can be used to excite water molecules in the plant tissue which causes cells to rupture and release the compounds trapped in the extracellular tissues of the plant material.

To confirm the presence of the desired components in the extract (i.e. the saponins and/or sapogenins), a variety of analytical techniques well known to those of skill in the art may be employed. Such techniques include, for example, chromatographic separation of organic molecules (e.g., gas chromatography or liquid chromatography), mass spectroscopy, or other spectroscopic techniques (such as infra-red, ultra-violet or nuclear magnetic resonance spectroscopy).

Determination of the Anti-Cancer Activity of the Compositions

Following preparation of a candidate composition, the anti-cancer activity of the composition can be measured and compared to that of the individual components. A number of tests familiar to a worker skilled in the art may be used to test the anti-cancer activity of the extracts, compositions, and formulations of the invention.

i) In vitro Testing

Initial determinations of the efficacy of the compositions of the present invention can be made using in vitro techniques.

For example, the cytotoxic activities of individual saponins or sapogenins for various cancer cell lines may be compared to compositions of the present invention using an assay for 3-(4,5-dimethylthiazol-2-yl)-2,5-diphenyltetrazolium bromide (MTT). Typically, in this technique, cancer cells are cultured and plated into a 96-well plate. The cells are then exposed to the candidate composition for 24 h. The medium is removed from the plates and replaced with MTT solution. The plates are analyzed and the number of viable cells in each well containing cells treated with the composition is calculated from the absorbance at 570 nm compared to untreated controls, or controls treated with one component of the composition alone. The means and standard deviation at each concentration for each agent are calculated using standard statistical methods.

Alternatively, the compositions can be tested by determining their ability to inhibit anchorage-independent growth of tumor cells. Anchorage-independent growth is known in the art to be a good indicator of tumorigenicity. In general, anchorage-independent growth is assessed by plating cells from an appropriate cancer cell-line onto soft agar and determining the number of colonies formed after an appropriate incubation period. Growth of cells treated with the candidate composition can then be compared with that of cells treated with an appropriate control (ie. cells treated with the saponin and/or sapogenin components alone) and with that of untreated cells.

Toxicity of the compositions can also be initially assessed in vitro using standard techniques. For example, human primary fibroblasts can be treated in vitro with the compositions and then tested at different time points following treatment for their viability using a standard viability assay, such as the trypan-blue exclusion assay. Cells can also be assayed for their ability to synthesize DNA, for example, using a thymidine incorporation assay, and for changes in cell cycle dynamics, for example, using a standard cell sorting assay in conjunction with a fluorocytometer cell sorter (FACS).

ii) In Vivo Testing

The ability of the compositions to inhibit tumor growth or proliferation in vivo can be determined in an appropriate animal model using standard techniques known in the art (see, for example, Enna, et al, Current Protocols in Pharmacology, J. Wiley & Sons, Inc., New York, N.Y.).

In general, current animal models for screening anti-tumor compounds are xenograft models, in which a human tumor has been implanted into an animal. Examples of xenograft models of human cancer include, but are not limited to, human solid tumor xenografts in mice, implanted by sub-cutaneous injection and used in tumor growth assays; human solid tumor isografts in mice, implanted by fat pad injection and used in tumor growth assays; experimental models of lymphoma and leukaemia in mice, used in survival assays, and experimental models of lung metastasis in mice.

For example, the compositions can be tested in vivo on solid tumors using mice that are subcutaneously grafted bilaterally with 30 to 60 mg of a tumor fragment on day 0. The animals bearing tumors are mixed before being subjected to the various treatments and controls. In the case of treatment of advanced tumors, tumors are allowed to develop to the desired size, animals having insufficiently developed tumors being eliminated. The selected animals are distributed at random to undergo the treatments and controls. Animals not bearing tumors may also be subjected to the same treatments as the tumor-bearing animals in order to be able to dissociate the toxic effect from the specific effect on the tumor. Chemotherapy generally begins from 3 to 22 days after grafting, depending on the type of tumor, and the animals are observed every day. The compositions of the present invention can be administered to the animals, for example, by bolus infusion. The different animal groups are weighed about 3 or 4 times a week until the maximum weight loss is attained, after which the groups are weighed at least once a week until the end of the trial.

The tumors are measured about 2 or 3 times a week until the tumor reaches a pre-determined size and/or weight, or until the animal dies if this occurs before the tumor reaches the pre-determined size/weight. The animals are then sacrificed and the tissue histology, size and/or proliferation of the tumor assessed.

For the study of the effect of the compositions on leukaemias, the animals are grafted with a particular number of cells, and the anti-tumor activity is determined by the increase in the survival time of the treated mice relative to the controls.

To study the effect of the compositions of the present invention on tumor metastasis, tumor cells are typically treated with the composition ex vivo and then injected into a suitable test animal. The spread of the tumor cells from the site of injection is then monitored over a suitable period of time.

In vivo toxic effects of the compositions can be evaluated by measuring their effect on animal body weight during treatment and by performing haematological profiles and liver enzyme analysis after the animal has been sacrificed.

TABLE 1
Examples of xenograft models of human cancer

| Cancer Model | Cell Type |
|---|---|
| Tumor Growth Assay | Prostate (PC-3, DU145) |
| Human solid tumor xenografts in mice (sub- | Breast (MDA-MB-231, MVB-9) |
| cutaneous injection) | |
| | Colon (HT-29) |
| | Lung (NCI-H460, NCI-H209) |
| | Pancreatic (ASPC-1, SU86.86) |
| | Pancreatic: drug resistant (BxPC-3) |
| | Skin (A2058, C8161) |
| | Cervical (SIHA, HeLa-S3) |
| | Cervical: drug resistant (HeLa S3-HU-resistanc |
| | Liver (HepG2) |
| | Brain (U87-MG) |
| | Renal (Caki-1, A498) |
| | Ovary (SK-OV-3) |
| Tumor Growth Assay | Breast: drug resistant (MDA-CDDP-S4, MDA- |
| | MB435-To.1) |
| Human solid tumor isografts in mice (fat | |
| pad injection) | |
| Survival Assay | Human: Burkitts lymphoma (Non-Hodgkin's) |
| | (raji) |
| Experimental model of lymphoma and | Murine: erythroleukemia (CB7 Friend retroviru |
| leukaemia in mice | induced) |
| Experimental model of lung metastasis in | Human: melanoma (C8161) |
| mice | Murine: fibrosarcoma (R3) |

Therapeutic Use of the Compositions

The compositions of the present invention are useful in the treatment of cancer in mammalian subjects, including humans.

Examples of cancers which may be may be treated or stabilized with the compositions of present invention include, but are not limited to leukaemia, carcinomas, adenocarcinomas, melanomas and sarcomas. Carcinomas, adenocarcinomas and sarcomas are also frequently referred to as “solid tumors,” examples of commonly occurring solid tumors include, but are not limited to, cancer of the brain, breast, cervix, colon, head and neck, kidney, lung, ovary, pancreas, prostate, stomach and uterus, non-small cell lung cancer and colorectal cancer.

The term “leukaemia” refers broadly to progressive, malignant diseases of the blood-forming organs. Leukaemia is typically characterized by a distorted proliferation and development of leukocytes and their precursors in the blood and bone marrow but can also refer to malignant diseases of other blood cells such as erythroleukaemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “sarcoma” generally refers to a tumor which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinpma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas and prostate.

Additional cancers encompassed by the present invention include, for example, Hodgkin's Disease, Non-Hodgkin's lymphoma, multiple myeloma, neuroblastoma, rhabdomyosarcoma, primary thrombocytosis, primary macroglobulinemia, small-cell lung tumors, primary brain tumors, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, mesothelioma and medulloblastoma

In one embodiment of the present invention, the compositions are used in the treatment or stabiisation of a cancer selected from the group of: pancreatic cancer, lung cancer, stomach cancer, esophagus cancer, colon and rectum cancer, brain cancer (glioma), ovary cancer, liver cancer, kidney cancer, larynx cancer, bone cancer, multiple myeloma, melanoma, breast cancer, prostate cancer, bladder cancer, cancer in body of uterus, oral cavity cancer, thyroid cancer, cervix cancer, testis cancer, non-Hodgkin's lymphoma, leukemia, Hodgkin's disease, skin cancer, and soft tissue cancer.

The compositions of the present invention can be used to treat or stabilise advanced and/or metastatic cancers, including the above cancers when in an advanced and/or metastatic stage. Such cancers are frequently drug resistant. The compositions may also be used to treat recurrent or refractory versions of the above-listed cancers.

Additional cancers encompassed by the present invention include, for example, primary and metastatic multi-drug resistant cancers of various tissues and/or organs, comprising, with no limitation being implied: muscle, bone or connective tissues, the skin, brain, lungs, sex organs, the lymphatic or renal systems, mammary or blood cells, liver, the digestive tract, pancreas and thyroid or adrenal glands. These pathological conditions can also include solid tumors, cancers of the ovary, breast, brain, prostate, colon, stomach, kidney or testicles, Kaposi's sarcoma, cholangioma, chorioma, neuroblastoma, Wilms' tumor, Hodgkin's disease, melanomas, multiple myelomas, lymphatic leukemias and acute or chronic granulocytic lymphomas.

The development of drug resistance is one of the major obstacles in the management of cancer. One of the traditional ways to attempt to circumvent this problem of drug resistance has been combination chemotherapy, which uses the differing mechanisms of action and cytotoxic potentials of multiple drugs. A drug that exhibits the ability to overcome multi-drug resistance could be employed as a chemotherapeutic agent either alone or in combination with other drugs. The potential advantages of using such a drug in combination with chemotherapy would be the need to employ fewer toxic compounds in the combination, cost savings, and an enhanced effect leading to a treatment regimen involving fewer treatments.

The present invention contemplates that the compositions in conjunction with one or more chemotherapeutic agents, may be used as part of a combination chemotherapy regimen to treat a subject having a multi-drug resistant cancer. Thus, the compositions according to the present invention can be used either alone or in combination with other pharmacologically active chemotherapeutic agents to treat cancers.

Pharmaceutical Formulations

The compositions of the present invention may be administered as pharmaceutical formulations with an appropriate pharmaceutically physiologically acceptable carrier, diluent, excipient or vehicle. The pharmaceutical formulation may also be formulated to contain the composition and one or more other chemotherapeutic agents for concurrent administration to a subject.

The pharmaceutical formulations of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrastemal injection or infusion techniques.

The pharmaceutical formulations may be in a form suitable for oral use, for example, as tablets, troches, lozenges, aqueous or oily suspensions, dispersible powders or granules, emulsion hard or soft capsules, or syrups or elixirs. Formulations intended for oral use may be prepared according to methods known to the art for the manufacture of pharmaceutical formulations and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed.

Pharmaceutical formulations for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present

Pharmaceutical formulations of the invention may also be in the form of oil-in-water emulsions. The oil phase may be a vegetable oil, for example, olive oil or arachis oil, or a mineral oil, for example, liquid paraffin, or it may be a mixture of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical formulations may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

Other pharmaceutical formulations and methods of preparing pharmaceutical formulations are known in the art and are described, for example, in “Remington: The Science and Practice of Pharmacy,” Gennaro, A., Lippincott, Williams & Wilkins, Philidelphia, Pa. (2000) (formerly “Remingtons Pharmaceutical Sciences”).

Non-Pharmaceutical Formulations

Non-pharmaceutical formulations are formulations that do not require pre-market drug approval from a governmental agency regulating the manufacture, use or sale of pharmaceuticals or biological products, for example, the United States Food and Drug Administration. A person of skill in the art will recognize that a formulation may exist as both a pharmaceutical as well as a non-pharmaceutical formulation. For example, a botanical formulation may be considered as both a pharmaceutical formulation and a non-pharmaceutical formulation. Non-pharmaceutical formulations may be formulated by using the same methods, in the same forms and with the same pharmacologically acceptable media or carriers as for pharmaceutical formulations (as described above).

The non-pharmaceutical formulation may be in the form of, for example, a nutraceutical composition, a food, a health food, a natural health product, a functional food, a nutritional supplement, a dietary supplement, an herbal supplement, an herb, an alternative medicine, or a naturopathic product. In one embodiment of the invention, the composition of the present invention is provided as a non-pharmaceutical formulation in a pharmacologically acceptable medium. The non-pharmaceutical formulations disclosed herein may typically be in the form of a tablet, a capsule, or an ointment

Administration and Dosage Protocols

In accordance with the present invention, a therapeutically effective amount of the composition or a formulation comprising the composition of the present invention is administered to a subject in order to treat cancer. The composition or a formulation comprising the composition may be administered in a manner consistent with conventional chemotherapeutic practice.

The dosage of the composition of the present invention to be administered will be dependent upon the type of cancer to be treated, the severity of symptoms experienced by the patient, and the size of the subject, and can be readily determined by a skilled practitioner. It is to be understood, however, that the dosage and frequency of administration may be adapted to the circumstances in accordance with known practices in the art, for the treatment of different cancers.

As an example, the therapeutically effective amount of each active component in the composition may be between about 0.01 mg and about 1000 mg, per kg of body weight. In one embodiment, the composition comprises a therapeutically effective dosage of Rh2 of between about 0.01 mg and about 1000 mg per kg of body weight. In another embodiment, the composition comprises a therapeutically effective dosage of aglycon protopanaxatriol of between about 0.01 mg and about 1000 mg per kg of bpdy weight. In a further embodiment, the composition comprises a therapeutically effective dosage of aglycon protopanaxadiol of between about 0.01 mg and about 1000 mg per kg of body weight.

Pharmaceutical Kits

The present invention additionally provides for therapeutic kits containing the composition of the present invention in pharmaceutical formulations for use in the treatment of cancer. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a subject.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a subject. Such an instrument may be an inhalant, syringe, pipette, forceps, measured spoon, eye dropper or any such medically approved delivery vehicle.

The kit may further comprise one or more other chemotherapeutic agents for administration to a subject in conjunction with the composition of the present invention.

To gain a better understanding of the invention described herein, the following examples are set forth. It should be understood that these examples are for illustrative purposes only. Therefore, they should not limit the scope of this invention in any way.

### EXAMPLES

The anti-cancer effect of compositions, comprising Rh2, aPPT, and aPPD, were compared to each of the individual components. Compositions were prepared by combining the individual components in the following amounts (% w/w):

| | Composition (1) | Composition (2) | Composition (3) |
|---|---|---|---|
| Rh2 | 7.3% | 14.3% | 41.2% |
| aPPT | 26.3% | 16.5% | 17.6% |
| aPPD | 66.4% | 69.2% | 41.2% |
| Total | 100% | 100% | 100% |

Preparation of Compositions: A process of preparing a composition comprising of Rh2, aPPD, and aPPT saponins and/or sapogenins from plants selected from the group consisting of panax ginseng, panax quinguefol and panax notoginseng, or a sapogenin source from some other plant, and proceeding according to the following steps:
(a) mixing the ginsenoside extract with water;
(b) (i) mixing the ginsenoside extract and water with a short-chain (1-5 carbon) alkali-metal alcoholate solution or a hydroxide-ethanol solution to produce a mixture; and
   (ii) placing the resultant mixture in a reaction tank so that the resultant mixture can undergo chemical reactions at a temperature between 240 and 300° C. and at a pressure between 3.5 and 8.4 MPa; or
(c) (i) alternatively, mixing the ginsenosides extract with ethanol;
   (ii) mixing the extract and ethanol with alkali-metal alcoholates solution to produce a mixture, and
   (iii) placing the resultant mixture in a reaction tank so that the resultant mixture can undergo chemical reactions at a temperature between 240 and 300° C. and at a pressure between 3.5 and 8.4 MPa; and
(d) after the reaction is completed, collecting an intermediate product of a mix of saponins and/or sapogenins from the ethanol mixture. Cytotoxicity was measured using a standard microculture tetrazolium assay (MTT) (Alley, M C et al, Cancer Research 48:589-601, 1988). Exponentially growing cultures of tumor cells, including multi-drug resistant cell lines, were used to make microtiter plate cultures. Cells were seeded at 1.2×10³ cells per well in 96-well plates, and grown overnight at 37° C. Test compounds were then added. Cells were treated for 24 hours. To determine the number of viable cells in each well, the MTT dye was added (3-(4,5-dimethylthiazol-2-yl)-2,5-diphenyltetrazolium bromide in saline) in accordance with the standard practice known in the art.

### Example 1

### Enhanced Effect of a Composition Comprising Rh2, aPPT, and aPPD on U87 Human Glioma Cells

Cultured human glioma tumor cells (U87) were treated with the saponins and sapogenins Rh2, aPPT, and aPPD, individually, at various concentrations. Similarly, the U87 cells were also treated with a composition comprising the saponins and sapogenins Rh2, aPPT, and aPPD. Composition 1 used in this example has a chemical profile of 7.3% Rh2, 26.3% aPPT, and 66.4% aPPD (% w/w). The cell viabilities were examined with MTT at 24 hours.

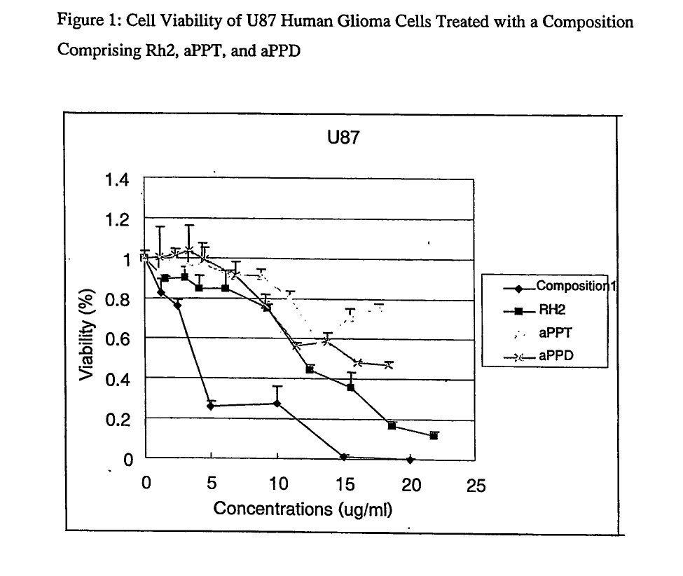

FIG. 1 demonstrates the enhanced effect of Composition 1 on human glioma cells (U87). As shown in the figure, 15 μg/mL of the composition killed nearly 100% (99%) of cancer cells, while similar concentrations of the individual components, Rh2, aPPT, and aPPD only killed 64%, 29%, and 52%, respectively. In this example, the result is a much improved efficacy and potency compared to each individual component at concentrations that are the same as the total of the composition.

### Example 2

### Enhanced Effect of a Composition Comprising Rh2, aPPT, and aPPD on B16 Mouse Melanoma Cells

Cultured mouse melanoma cells (B16) were treated with the saponins and sapogenins Rh2, aPPT, and aPPD, individually, at various concentrations. Similarly, the B16 cells were also treated with a composition of the present invention comprising the saponins and sapogenins Rh2, aPPT, and aPPD. Composition 2 used in this example has a chemical profile of 14.3% Rh2, 16.5% aPPT, and 69.2% aPPD (% w/w). The cell viabilities were examined with MTT at 24 hours.

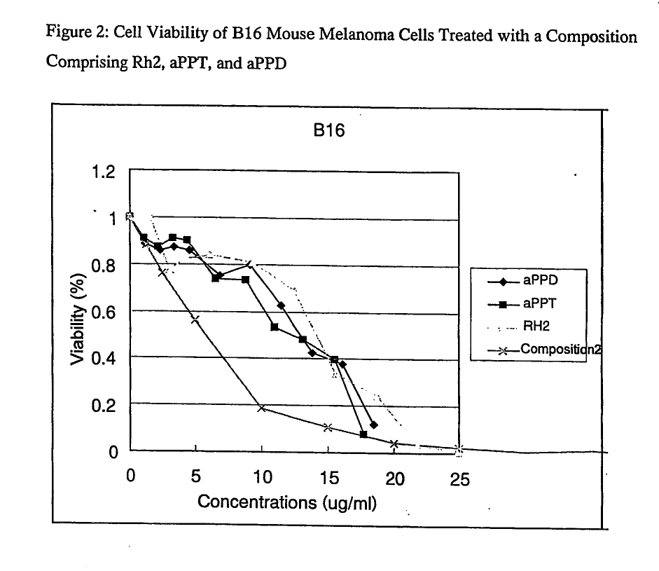

FIG. 2 demonstrates the enhanced effect of Composition 2 on mouse melanoma cell B16. As shown in the figure, 10 μg/mL of the composition killed 81% of cancer cells, while similar concentrations of Rh2, APPT, or aPPD only killed 56%, 47%, and 44%, respectively. In this example, the result is a much improved efficacy and potency compared to each individual component at concentrations that are the same as the total of the composition.

### Example 3

### Enhanced Effect of a Composition Comprising Rh2, aPPT, and aPPD on MCF7 Human Breast Cancer Cells

Cultured human breast cancer cells (MCF7) were treated with the saponins and sapogenins Rh2, aPPT, and aPPD, individually, at various concentrations. Similarly, the MCF7 cells were also treated with a composition of the present invention comprising the saponins and sapogenins Rh2, aPPT, and aPPD. Composition 3 used in this example has a chemical profile of 41.2% Rh2, 17.6% aPPT, and 41.2% aPPD (% w/w). The cell viabilities were examined with MTT at 24 hours.

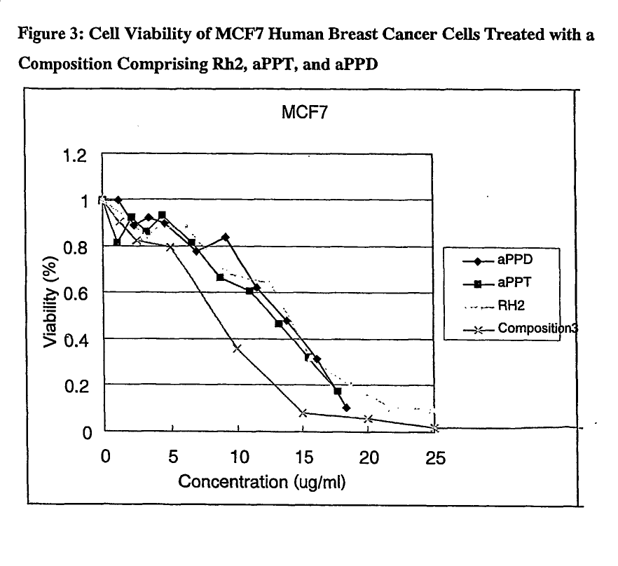

FIG. 3 demonstrates the enhanced effect of Composition 3 on human breast cancer cell MCF7. As shown in the figure, 15 μg/mL of the composition killed 92% of cancer cells, while similar concentrations of Rh2, aPPT, or aPPD only killed 68%, 68%, and 69%, respectively. In this example, the result is a much improved efficacy and potency compared to each individual component at concentrations that are the same as the total of the composition.

### Example 4

### Efficacy of a Composition Comprising Rh2, aPPT, and APPD on Multi-Drug Resistant/Advanced Cancer Cells

Drug resistant pancreatic cancer cells (BXCP-3) and (CAPAN-1) were treated with a composition comprising the saponins and sapogenins Rh2, aPPT, and aPPD. Composition 1 used in this example has a chemical profile of 7.3% Rh2, 26.3% aPPT, and 66.4% aPPD (% w/w). The cell viabilities were examined with MTT at 24 hours.

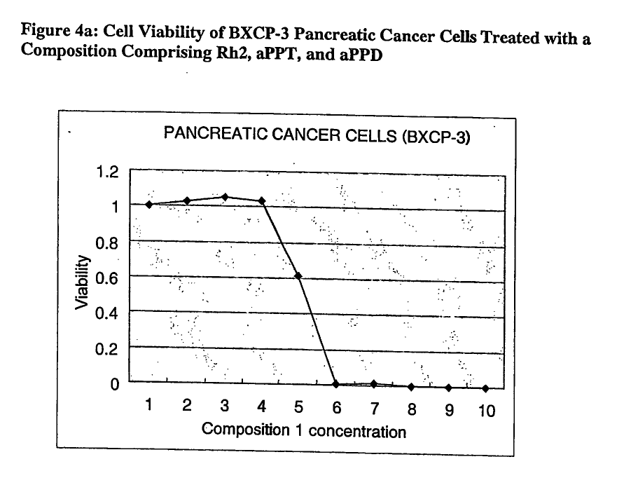

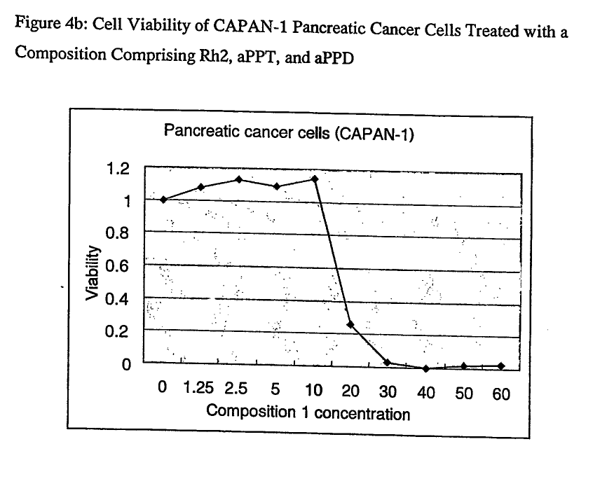

FIG. 4a shows the effect of the various concentrations of Composition 1 on the drug-resistant cell line (BXCP-3). FIG. 4b shows the effect of the various concentrations of Composition 1 on the drug-resistant cell line (CAPAN-1). It is clear that both cell lines were responsive to the composition.

### Example 5

### Anti-Cancer Effect of a Composition Comprising Rh2, aPPT, and aPPD on Various Cancer Cells

The efficacy of Composition 1 in inhibiting the growth of various cancer cells is shown in Table 2.

TABLE 2

| | | Minimum | |
|---|---|---|---|
| | | concentrations for | |
| Cancer cell lines | Cancer Type | 100% cell killing | |
| MCF7/adr | Breast cancer | 40 ug/ml | Drug |
| | | | resistant |
| MDA435LCC6M | Breast cancer | 40 ug/ml | Drug |
| | | | resistant |
| MCF-7/c3 | Breast cancer | 40 ug/ml | Caspase- |
| | | | 3 positive |
| 9L | Brain tumor | 25 ug/ml | |
| SF188 | Brain tumor | 20 ug/ml | |
| LNCaP | Prostate tumor | 25 ug/ml | |
| PC3 | Prostate tumor | 40 ug/ml | |
| HCT15 | Intestinal & Gastric | 40 ug/ml | |
| | tumor | | |
| Keto | Intestinal & gastric | 35 ug/ml | |
| | tumor | | |
| H460 | Lung cancer | 40 ug/mi | |

### Example 6

### In Vivo Cancer Inhibitory Effect of Composition 4

Composition 4:

| | |
|---|---|
| Rh2 | 8% |
| Rg3 | 2% |
| Sapogenins | 70% |
| Unknown ginsenosides | 20% |

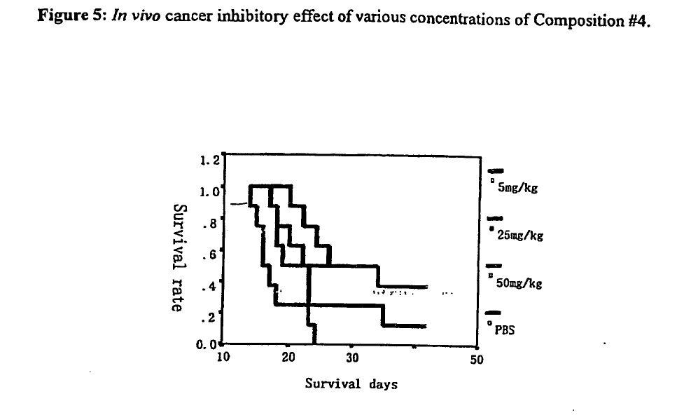

To test the anticancer activity of Composition 4 in vivo, an animal malignant brain tumor model was utilized. Tumor cells were implanted into rat brain and three doses of Composition 4 were given orally on day 10 postimplantation when the tumor had been established. As shown in FIG. 5, after 2×5 days of treatment with Composition 4, there are significant difference in survival rates of animals between the treatment group and control. Especially for animals with 25 mg/kg and 50 mg/kg, 40% of animals survived longer than 42 days comparing to the average of 18 days in control animals. Furthermore, more than 50% of survived animals were found completed tumor regression by autopsy.

### Example 7

### Toxicological Profile of Composition 4

Formal acute and long term toxicity tests of Composition 4 were conducted. Briefly, for acute toxicity test, rats were orally given 4000 mg/kg Composition 4 once. It was found that treated animals appeared quiet and less active with eyes closed 30 min after the treatment. The drowsiness disappeared in 4 hours and the activity, diet and water intake returned to normal. No animal died during 7 observation days and autopsy did not show any abnormality in heart, liver, spleen, lung, kidney, stomach and intestine. For the long term toxicity test, animals were orally given 333.3 mg/kg, 131.0 mg/kg, and 51.5 mg/kg Composition 4 as high (H), medium (M) and low (L) dosages, respectively. The treatment was daily and lasted 8 weeks plus 2 weeks post-treatment observation period. Transient hypertrophy in the prostate (187%) and ovary (124%) were found in group H animals in 24 hours. Similar hypertrophy in the prostate (156%) was found in group M. However, no hypertrophy was found after 2 weeks. Other minor toxic reactions were only seen in group H, including drowsiness during week 3 and 4 (back to normal thereafter) and less weight gain (no statistic significance) in later stage of the treatment (week 4-9). The blood routine and organ histology examinations did not shown any abnormality in all the three groups.

Acute toxicity tests of Composition 4 were conducted. The results are briefly summarized as follows.

I. Blood Vessel Irritation Test:
Composition 4 was given i.v. via the ear vein in rabbits (Japanese Big-ear) at the dose of 5 ml/kg (1 mg/ml) daily for 7 days consecutively. No noticeable changes were seen in the injection region. Microscopic examination on tissue sections of the injection region did not found venous congestion, edema or infiltration of inflammatory cells.
Conclusion: Composition 4 does not cause irritation to the blood vessels.
   II. Endotoxin Test:
Limulus Amebocyte Lysate (LAL) interference test was performed to determine the levels of endotoxin in Composition 4. The minimum detectable level of endotoxin was 0.125 EU/ml-0.25 EU/ml for the LAL test kits utilized. No interference was detected with Composition 4 at the dilutions of 1 mg/ml, 0.5 mg/ml, 0.25 mg/ml and 0.125 mg/ml.
Conclusion: The endotoxin level is less than 1 EU in 1 mg of Composition 4.

III. Hemolysis Test
Rabbit whole blood was used to mix with dilutions of Composition 4 and spectrophotometrical absorbance of the supernatant was measured at the wavelength of 545 nm to determine hemolysis. Saline diluted Composition 4 (Composition 4: saline=3:1, final concentration: 1 mg/ml) caused hemolytic effect was less than 1% (normal value: <5%). However, undiluted Composition 4 (4 mg/ml) caused minor hemolysis (51%).
Conclusion: No hemolysis effect can be detected when Composition 4 is used in 1:3 saline diluted solution.

IV. Allergy Test
The allergy test was performed in genie pigs. 0.5 ml of Composition 4 was given i.p. every second day for 6 days (3 times). In addition, 1 ml Composition 4 was given i.v. to the same animal on day 14 and day 21. Typical allergy responses such as coughing, sneezing, skin/hair and respiratory reactions, etc. were monitored in 15 minutes following the injection.
Conclusion: Composition 4 does not cause allergy.

V. LD50 of Composition 4
Five groups of mice were i.v. injected once with 5 different doses of Composition 4 (from 83.5 mg/kg to 160.0 mg/kg). The animals were observed 7 days following the injection and the LD50 was determined using Bliss method. Severe toxicity and death were seen in animals with the dosages of 136 mg/kg and 160 mg/kg. Reducing dosages significantly reduced toxicity.
Conclusion: The LD50 of Composition 4 i.v. injection is 121.9 mg/kg with a 95% confidence range of 105.0-141.6 mg/kg.

### Example 8

### Informal Phase I Clinical Trial

An informal phase I clinical trial was performed in Liaoning Provincial TCM Research Institute. Forty patients of various types of malignant solid tumors who had failed with first line treatment or refused for conventional treatment were enrolled. The patients suffered from the following cancers: lung (10 patients), liver (6 patients), breast (5 patients), R & C (4 patients), uterus (3 patients), pancreatic (2 patients), nose-throat (1 patient), eosophagus (1 patient), bile duct (1 patient), stomach (1 patient), testicular (1 patient), kidney (1 patient), bladder (1 patient), ureter (1 patient), mouth (1 patient) and hepatosorrhesis (1 patient). The 40 patients with average age of 56.1±11.5 yrs were treated with 200 mg/day Composition 4 orally for average duration of 53.7±25.4 days. The primary objective was to test for the toxicity of Composition 4 measured by the symptoms of nausea, vomiting, stomatitis, hair loss, nervous system symptoms and skin abnormality. None of the above symptoms were reported in any patients observed. The toxicity was then concluded as grade 0.

Although it was not the primary goal of this trial, preliminary observations of efficacy were very encouraging. It was reported that 75% of the patients treated with Composition 4 have demonstrated improvement in their symptoms to various degrees and some of them had shown significant tumor regression.

### Case Examples

Case 1: Female, 58 yr, Lower bile duct cancer (3.0×2.6 cm)
Symptoms: weight loss, nausea, dizziness, fatigue, upper abdominal pain, icterus, etc.
Treatment: 114 days, 200 mg/day.
Improvement: Tumor size reduced to 1.8×1.4 cm. Above symptoms disappeared. Feeling well by herself.
   Case 2: Female, 74 yr, Esophagus cancer (umbrella type), 6.0 cm long (x-ray image).
Symptoms: difficulty in swallowing, pain, weight loss, frequent hiccups.
Treatment: 110 days, 200 mg/day.
Improvement: Tumor size reduced to 1.5 cm, no difficulty in swallowing, much less hiccups, no pain. Feeling well by herself.
   Case 3: Male, 66 yr, Pancreatic cancer 4.5×4.5×3.7 cm.
Symptoms: fatigue, dizziness, upper abdominal and liver pain, abnormal high GPT, GOT, and blood sugar.
Treatment: 90 days, 200 mg/day.
Improvement: Much less pain. GPT, GOT, blood sugar all returned to normal.

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A composition comprising an activity-enhancing amount of one or more saponins in combination with one or more sapogenins, and having an enhanced anti-cancer activity.

2. The composition according to claim 1, wherein the saponins and sapogenins are selected from the group comprising: Rh2, Rg3, aglycon protopanaxatriol, and aglycon protopanaxadiol.

3. The composition according to claim 2, comprising about 70% sapogenins, about 8% Rh2, and about 2% Rg3.

4. The composition according to claim 2, comprising between about I-90% each of Rh2, aglycon protopanaxatriol, and aglycon protopanaxadiol.

5. The composition according to claim 4, comprising between about 1-50% of Rh2, between about 5-40% of aglycon protopanaxatriol, and between about 5-75% of aglycon protopanaxadiol.

6. The composition according to claim 5, comprising between about 5-40% of Rh2, between about 5-40% of aglycon protopanaxatriol, and between about 10-70% of aglycon protopanaxadiol.

7. The composition according to claim 1, wherein one or more of said saponins and sapogenins are extracted from plant material.

8. The composition according to claim 7, wherein said plant material is derived from one or more plant from the genus Panax.

9. The composition according to claim 8, wherein said plant is Panax ginseng.

10. The composition according to claim 8, wherein said plant is Panax quinquefolium.

11. The composition according to claim 8, wherein said plant is Panax notoginseng.

12. The composition according to claim 1, wherein one or more of said saponins and sapogenins are synthetic.

13. A pharmaceutical formulation for the treatment of cancer, comprising a therapeutically effective amount of the composition according to claim 1, and a pharmacologically acceptable carrier.

14. A non-pharmaceutical formulation for the treatment of cancer, comprising a therapeutically effective amount of the composition according to claim 1, and a pharmacologically acceptable carrier.

15. The formulation according to claim 13, wherein the formulation is in an orally administrable form.

16. The formulation according to claim 13, wherein the formulation is in an injectable form.

17. The formulation according to claim 13, wherein the formulation is in a topically applicable form.

18. The formulation according to claim 13, wherein said therapeutically effective amount comprises a dosage of between 0.01 mg to 1000 mg of Rh2 per kg bodyweight per day.

19. The formulation according to claim 13, wherein said therapeutically effective amount comprises a dosage of between 0.01 mg to 1000 mg of aglycon protopanaxatriol per kg bodyweight per day.

20. The formulation according to claim 13, wherein said therapeutically effective amount comprises a dosage of between 0.01 mg to 1000 mg of aglycon protopanaxadiol per kg bodyweight per day.

21. Use of the composition according to claim 1 for the treatment of cancer in a mammal.

22. The use according to claim 21, wherein said cancer is selected from the group consisting of glioma tumor, melanoma, breast cancer, pancreatic cancer, brain tumor, intestinal and gastric cancers, prostate cancer, and lung cancer.

23. The use according to claim 21, wherein said cancer is selected from the group consisting of stomach cancer, esophagus cancer, colon and rectum cancer, ovary cancer, liver cancer, kidney cancer, larynx cancer, bone cancer, multiple myeloma, bladder cancer, cancer in body of uterus, oral cavity cancer, thyroid cancer, cervix cancer, testis cancer, non-Hodgkin's lymphoma, leukemia, Hodgkin's disease, skin cancer, and soft tissue cancer.

24. The use according to claim 21, wherein said cancer is a multi-drug resistant cancer.

25. The use according to claim 24, wherein said multi-drug resistant cancer is a primary cancer selected from the group consisting of cancers of muscle, bone or connective tissues, the skin, brain, lungs, sex organs, the lymphatic or renal systems, mammary or blood cells, liver, the digestive tract, pancreas and thyroid or adrenal glands, including solid tumors, cancers of the ovary, breast, brain, prostate, colon, stomach, kidney or testicles, Kaposi's sarcoma, cholangioma, chorioma, neuroblastoma, Wilms' tumor, Hodgkin's disease, melanomas, multiple myelomas, lymphatic leukemias and acute or chronic granulocytic lymphomas.

26. The use according to claim 24, wherein said multi-drug resistant cancer is a recurrent cancer selected from the group consisting of pancreatic cancer, lung cancer, stomach cancer, esophagus cancer, colon and rectum cancer, brain cancer, ovary cancer, liver cancer, kidney cancer, larynx cancer, bone cancer, multiple myeloma, melanoma, breast cancer, prostate cancer, bladder cancer, cancer in body of uterus, oral cavity cancer, thyroid cancer, cervix cancer, testis cancer, non-Hodgkin's lymphoma, leukemia, Hodgkin's disease, skin cancer, and soft tissue cancer.

27. The use according to claim 21, wherein said composition is used in combination with one or more other chemotherapeutic agents.

28. The use according to claim 21, wherein said mammal is a human.

29. The use of a composition according to claim 1 in the manufacture of a medicament for the treatment of cancer.

30. A pharmaceutical kit for the treatment of cancer in a mammal comprising the composition according to claim 1 and one or more containers.

31. The formulation according to claim 14, wherein the formulation is in an orally administrable form.

32. The formulation according to claim 14, wherein the formulation is in an injectable form.

33. The formulation according to claim 14, wherein the formulation is in a topically applicable form.

34. The formulation according to claim 14, wherein said therapeutically effective amount comprises a dosage of between 0.01 mg to 1000 mg of Rh2 per kg bodyweight per day.

35. The formulation according to claim 14, wherein said therapeutically effective amount comprises a dosage of between 0.01 mg to 1000 mg of aglycon protopanaxatriol per kg bodyweight per day.

36. The formulation according to claim 13, wherein said therapeutically effective amount comprises a dosage of between 0.01 mg to 1000 mg of aglycon protopanaxadiol per kg bodyweight per day.
